(19) United States
(12) Patent Application Publication
(10) Pub. No.: US 2019/0038179 A1
(43) Pub. Date: Feb. 7, 2019

(54) METHODS AND APPARATUS FOR IDENTIFYING BREATHING PATTERNS

(71) Applicant: Intel Corporation, Santa Clara, CA (US)

(72) Inventors: Cagri Tanriover, Istanbul (TR); Hector Cordourier Maruri, Guadalajara (MX); Paulo Lopez Meyer, Zapopan (MX); Asli Arslan Esme, Istanbul (TR)

(21) Appl. No.: 15/669,137

(22) Filed: Aug. 4, 2017

(51) Int. Cl. A61B 5/08; A61B 5/00; A61B 7/00

(52) U.S. Cl. CPC A61B 5/0816; A61B 5/7405; A61B 5/742; A61B 5/7455; A61B 5/746; A61B 5/7278; A61B 5/7267; A61B 5/725; A61B 7/003; A61B 5/0022; A61B 5/0803; A61B 5/6803; A61B 5/486

(57) ABSTRACT

Methods and apparatus for identifying breathing patterns are disclosed herein. An example wearable device includes a sensor positioned to generate vibration signal data from a nasal bridge of a user, a breathing phase detector to identify a first and second breathing phases based on the vibration signal data, a phase timing calculator to calculate a first time period for the first breathing phase and a second time period for the second breathing phase, a breathing pattern detector to generate a breathing pattern metric based on the first and second time periods, a breathing activity detector to identify a breathing activity associated with the vibration signal data based on the breathing pattern metric, and an alert generator to activate an output device to generate at least one of an audible, tactile, or visual alert based on at least one of the breathing activity and a change associated with the breathing activity.

### FIELD OF THE DISCLOSURE

This disclosure relates generally to monitoring breathing activity in subjects and, more particularly, to methods and apparatus for identifying breathing patterns.

### BACKGROUND

A breathing cycle includes an inspiration phase (e.g., inhalation) and an expiration phase (e.g., exhalation). The breathing cycle also includes brief pauses between the inspiration phase and the expiration phase during the breathing cycle and the expiration phase and the inspiration phase as a new breathing cycle begins. Breathing cycle patterns typically change relative to an activity being performed by a subject, such as exercising, smelling an object, or relaxing.

### BRIEF DESCRIPTION OF THE DRAWINGS

FIG. 1 illustrates an example system constructed in accordance with the teaching disclosed herein and including a breathing data collection device and a processor for identifying breathing patterns.

FIG. 2 is a block diagram of an example implementation of the breathing pattern analyzer of FIG. 1.

FIG. 3 is a graph illustrating example nasal bridge vibration data.

FIG. 4 is a graph illustrating a root-mean-square envelope profile of the example nasal bridge vibration data of FIG. 3.

FIG. 5 is a flowchart representative of example machine readable instructions that may be executed to implement the example system of FIGS. 1 and/or 2.

FIG. 6 illustrates an example processor platform that may execute the example instructions of FIG. 5 to implement the example system of FIGS. 1 and/or 2.

The figures are not to scale. Wherever possible, the same reference numbers will be used throughout the drawing(s) and accompanying written description to refer to the same or like parts.

### DETAILED DESCRIPTION

A breathing cycle includes an inspiration phase in which air is drawn into a subject's lung and an expiration phase in which air is exhaled. The breathing cycle also includes a pause between the inspiration phase and the expiration phase before air is exhaled by the subject (i.e., an inspiration pause) and a pause between the expiration phase and the inspiration phase before air is inhaled by the subject (i.e., an expiration pause). Duration and frequency of the inspiration phase, the inspiration pause, the expiration phase, and/or the expiration pause can be affected by one or more activities performed by the subject. For example, when the subject is exercising, a frequency of the breathing cycles may increase and durations of the inspiration phases and expiration phases may be shortened as compared to when the subject is relaxed. As another example, when the subject smells an object such as a flower, a duration of the inspiration phase may be longer than a duration of the expiration phase.

Although some medical instruments monitor subject breathing and obtain data that can be used to identify breathing patterns, such instruments are generally limited to use in healthcare settings such as a hospital, often require the subject to be bed-bound to monitor the subject's breathing over time, and/or are generally uncomfortable or obtrusive for the subject. Further, wearable devices targeted for everyday use, such as fitness bands, do not provide for real-time and/or accurate detection of breathing patterns for the subject wearing the device. Moreover, such wearable devices do not account for differences in breathing data that arise based on whether the subject is breathing orally (i.e., primarily through his or her mouth) or nasally (i.e., primarily through his or her nose). However, whether a subject is breathing nasally or orally during an activity such as quiet breathing (e.g., a relaxed, normal breathing state for the subject, idle breathing) can be indicative of, for example, the subject's health.

In examples disclosed herein, breathing data is derived from nasal bridge vibrations that are generated as the subject breathes. In examples disclosed herein, a subject wears a wearable, head-mounted device such as eyeglasses that include one or more piezoelectric sensors coupled thereto. When the subject is wearing the eyeglasses, the sensor(s) are disposed proximate to the bridge of the subject's nose. As the subject breathes (e.g., inhales and exhales), the piezoelectric sensor(s) respond to vibrations and/or movement caused by the breathing (collectively and/or individually referred to here as vibrations) and produce corresponding electrical signal(s) that can be analyzed to identify breathing patterns in the signal data.

Example systems and methods disclosed herein analyze breathing data collected by a wearable (e.g., nasal bridge vibration data collected by sensors proximate to a subject's nose, sound data, etc.) to differentiate between different breathing activities performed by the subject, such as quiet breathing, smelling, breathing during exercise, etc. Some examples identify breathing patterns based on durations of peaks (e.g., inflection points) in the signal data indicative of, for example, inspiration or expiration phases. Some examples generate one or more breathing pattern metrics using the respective durations of the inspiration phase, the expiration phase, the inspiration pause, or the expiration pause. Some examples perform a rule-based analysis of the breathing pattern metrics to identify the breathing activities associated with the breathing data. In some examples, the subject's breathing activities are identified by a machine learning algorithm that is trained to classify breathing activities based on features of the signal data. Some examples identify frequency characteristics of the nasal bridge vibration data to determine if the subject is breathing orally or nasally.

Some disclosed examples analyze the subject's breathing data in substantially real-time via, for example, a processor associated with (e.g., carried by) the wearable device (e.g., the eyeglasses). Some disclosed examples analyze the subject's breathing data via a processor of a user device that is different from the wearable device that collects the data. For instance, the processor of a smartphone and/or other wearable such as a watch or the like. Other examples export the data to one or more cloud-based device(s) such as server(s), processor(s), and/or virtual machines to perform the analysis. Based on the determination of the subject's breathing patterns and/or the identification of breathing activities, disclosed examples can provide feedback to the subject in the form of, for example, alerts or notifications. Some disclosed examples activate output devices such as the wearable device that collects the data, another wearable device (e.g., a smartwatch), and/or, a non-wearable user device (e.g., a smartphone) to provide the alert(s) and/or notification(s) to the subject. The alert(s) can include tactile, visual, and/or audio alerts. For example, the notifications can include recommendations for more efficient breathing during exercise based on the analysis of the subject's breathing data, instructions for the subject to further quiet his or her breathing during mediation by increasing the duration of his or her inhalations and exhalations, instructions to direct exertion (e.g., if a potential health issue is detected, instructions to increase exertion to research a desired activity level (e.g., an optimal fat burning level)), etc.

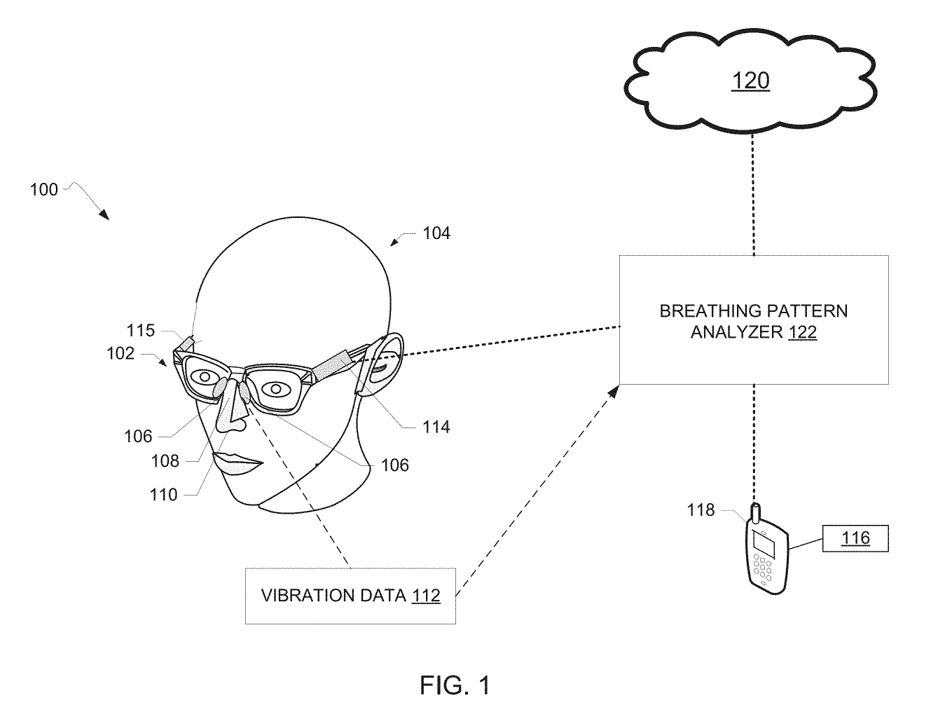

FIG. 1 illustrates an example system 100 constructed in accordance with the teachings of this disclosure for identifying breathing pattern(s) in a subject or user (the terms “user” and “subject” are used interchangeably herein and both refer to a biological creature such as a human being). The example system 100 includes a wearable device 102 to be worn by a user 104. In the example of FIG. 1, the wearable device 102 includes eyeglasses worn by the user 104. However, the wearable device 102 can include other wearables, such as a mask or a nasal strip.

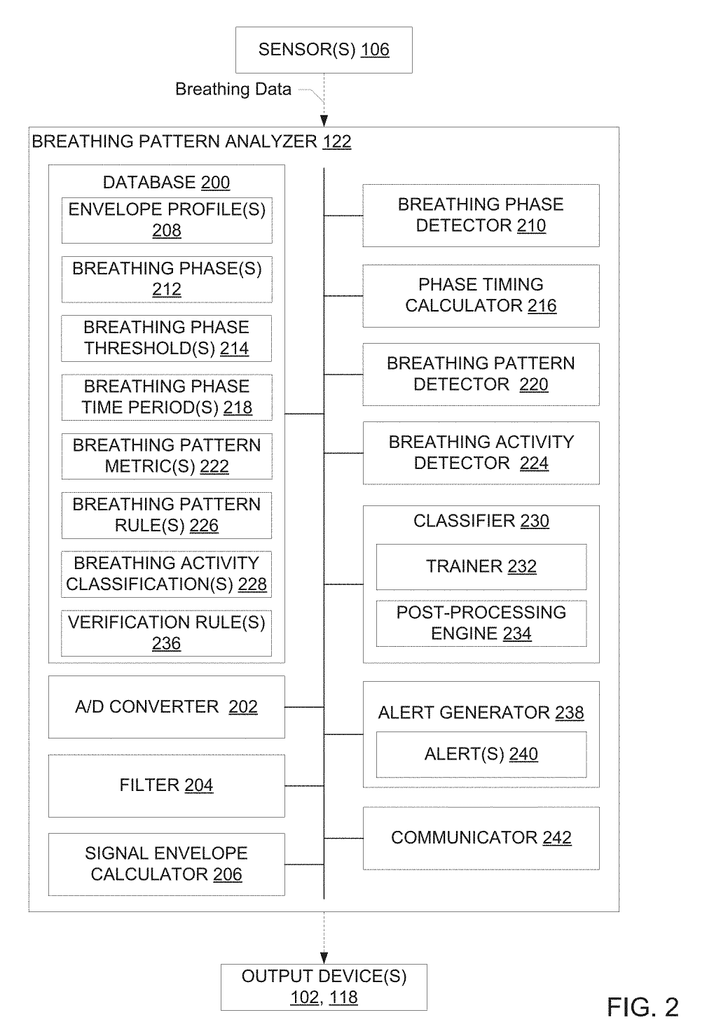

The wearable device 102 includes one or more sensors 106 coupled to the wearable device 102. In the example of FIG. 1, the sensor(s) 106 are piezoelectric sensor(s). The sensor(s) 106 are coupled to the wearable device 102 such that when the user 104 wears the wearable device 102, the sensor(s) 106 are disposed proximate to a bridge 108 of a nose 110 of the user 104. For example, the sensor(s) 106 can be coupled to nose pads of the wearable device 102. As the user 104 inhales and exhales, the sensor(s) 106 detect vibrations of the nasal bridge 108 due to the flow of air in and/or out of the user's nose 110. In some examples, the sensor(s) 106 detect vibrations of the nasal bridge as the user breathes in and out of his or her mouth. In particular, the piezoelectric sensor(s) 106 deform and generate electrical signal data based on the vibrations of the nasal bridge 108 during breathing. The sensor(s) 106 can measure the nasal bridge vibrations for a period of time (e.g., whenever the user 104 is wearing the wearable device 102, for a specific duration (e.g., not always on when the user is wearing the device 102), etc.). In other examples, the sensor(s) 106 measure vibrations at other portions of the user's body (e.g., the user's temples, the user's forehead). The wearable device 102 can include additional or fewer sensor(s) 106 than illustrated in FIG. 2. Also, the wearable device 102 can include other types of sensor(s) 106 and/or other means for generating the breathing data (e.g., via sound recordings). As used herein, breathing data is defined to include nasal bridge vibration data with or without other types of data such as sound data collected by other sensors.

The example system 100 of FIG. 1 includes one or more semiconductor based processors to store breathing data 112 (e.g., nasal bridge vibration data) generated by the sensor(s) 106, process the breathing data 112 generated by the sensor(s) 106, and/or generate one or more outputs based on the processing of the breathing data. For example, as illustrated in FIG. 1, a processor 114 is coupled (e.g., mounted) to the wearable device 102. Also, the wearable device 102 of FIG. 1 includes a battery 115 to provide power to the processor 114 and/or other components of the wearable device 102.

In other examples, the processor is separate from the wearable device 102. For example, the sensor(s) 106 can transmit the vibration data 112 to a processor 116 of a user device 118 such as a smartphone or another wearable (e.g., a smart watch). In other examples, the sensor(s) 106 can transmit the vibration data 112 to cloud-based device 120 (e.g., one or more servers, processor(s), and/or virtual machine(s)). The dotted lines extending from the breathing pattern analyzer 122 in FIG. 1 demarcate the different locations for the analyzer 122 (e.g., on the wearable 102, in the cloud 120, and/or in a wearable or non-wearable user device 118). Appropriate communication paths (e.g., via WiFi, cellular, Bluetooth and/or other communication protocols supported by the locations of the analyzer 122).

In some examples, the processor 114 of the wearable device 102 is communicatively coupled to one or more other processors. In such examples, the sensor(s) 106 can transmit the vibration data 112 to the on-board processor 114 of the wearable device 102. The on-board processor 114 can then transmit the vibration data 112 to the processor 116 of the user device 118 and/or the cloud-based device 120. In some such examples, the wearable device 102 (e.g., the sensor(s) 106 and/or the on-board processor 114) and the processor(s) 116, 120 are communicatively coupled via one or more wired connections (e.g., a cable) or wireless connections (e.g., cellular, Wi-Fi, or Bluetooth connections).

In the example system 100 of FIG. 1, the vibration data 112 is processed by a breathing pattern analyzer 122. The example breathing pattern analyzer 122 can be implemented by software executed on the processor 114 of the wearable device 102, the processor 116 of the wearable or non-wearable user device 118, and/or the cloud-based device 120. In some examples, one or more components of the example breathing pattern analyzer 122 are implemented by the on-board processor 114 of the wearable device 102 and one or more other components are implemented by the processor 116 of the user device 118 and/or the cloud-based device 120.

In the example system 100 of FIG. 1, the breathing pattern analyzer 122 serves to process the breathing data generated by the sensor(s) 106 to identify breathing patterns and/or breathing activities of the user 104. In the example system 100, the sensor(s) 106 collect the breathing data including nasal bridge vibration data 112 as the user 104 breathes. The breathing pattern analyzer 122 receives and processes the nasal bridge vibration data 112 collected by the sensor(s) 106. In some examples, the breathing pattern analyzer 122 receives the nasal bridge vibration data 112 and/or other breathing data in substantially real-time (e.g., near the time the data is collected). In other examples, the breathing pattern analyzer 122 receives the nasal bridge vibration data 112 and/or other breathing data at a later time (e.g., periodically and/or aperiodically based on one or more settings but sometime after the breathing has occurred (e.g., seconds, minutes, hours, days, etc. later). The breathing pattern analyzer 122 can perform one or more operations on the breathing data such as filtering the raw signal data, removing noise from the signal data, converting the signal data from analog data to digital data, and/or analyzing the data. For example, the breathing pattern analyzer 122 can convert the breathing data from analog to digital data at the on-board processor 114 and the digital data can be analyzed by on-board processor 114 and/or by one or more off-board processors, such as the processor 116 of the user device 124 and/or the cloud-based device 120.

The example breathing pattern analyzer 122 analyzes the breathing data from the user 104 to determine the user's breathing pattern. For example, the breathing pattern analyzer 122 identifies different vibration levels (e.g., amplitude levels) in the nasal bridge vibration data 112 and compares the vibration levels to particular vibration thresholds (the threshold levels may be predefined, static, and/or variable/adjustable to the user and/or historical data). Based on the comparisons, the breathing pattern analyzer 122 detects breathing phases (e.g., inspiration phase, inspiration pause, expiration phase, expiration pause) and determines the duration of each breathing phase. In some examples, the breathing pattern analyzer 122 generates one or more breathing pattern metrics based on the breathing phase durations. The example breathing pattern analyzer 122 identifies the user's breathing pattern based on the respective durations of the breathing phases, the breathing pattern metric(s), and/or one or more particular (e.g., predefined) rules. In some examples, the breathing pattern analyzer 122 uses the metric(s) and/or rule(s) to identify breathing activities being performed by the user 104, such as smelling or quiet breathing. In some examples, the breathing pattern analyzer 122 identifies the breathing activities using machine learning algorithms.

In some examples, the breathing pattern analyzer 122 generates one or more outputs based on the user's breathing pattern and/or the breathing activity. The outputs can include, for example, alerts or notifications to the user that provide for monitoring of the user's breathing and/or recommendations for the user to adjust his or her breathing and/or activities generating the breathing pattern. The alerts or notifications can be presented via the wearable device 102 (e.g., in the form of vibrations, sounds, visual signals, etc.) and/or the user device 118 (e.g., in the form of vibrations, visual, and/or audio alerts).

FIG. 2 is a block diagram of an example implementation of the example breathing pattern analyzer 122 of FIG. 1. As mentioned above, the example breathing pattern analyzer 122 is constructed to identify breathing patterns of a user (e.g., the user 104 of FIG. 1) and to detect breathing activities (e.g., smelling, quiet breathing). In the example of FIG. 2, the breathing pattern analyzer 122 is implemented by one or more of the processor 114 of the wearable device 102 of FIG. 1, the processor 116 of the user device 118, and/or cloud-based devices 120 (e.g., the server(s), processor(s), and/or virtual machine(s) 120 of FIG. 1). In some examples, some of the breathing data analysis is implemented by the breathing pattern analyzer 122 via a cloud-computing environment and one or more other parts of the analysis is implemented by the processor 114 of the wearable device 102 and/or the processor 116 of the user device 118.

In some examples, the location(s) at which the analysis is performed by the breathing pattern analyzer 122 is based on whether the analysis is to be performed in substantially real-time as the breathing data (e.g., the nasal bridge vibration data 112) is being generated or whether the analysis is to be performed at a later time. For example, if the analysis is to be performed in substantially real-time as the breathing data (e.g., the nasal vibration data 112) is being generated, the analysis may be performed at the processor 114 of the wearable device 102. In other examples, if the analysis is to be performed at a later time and/or if the breathing data (e.g., the nasal bridge vibration data 112) is to be transferred to the breathing pattern analyzer 122 at a later time, then the analysis may be performed at the processor 116 of the user device 118.

The example breathing pattern analyzer 122 of FIG. 2 includes a database 200. In other examples, the database 200 is located external to the breathing pattern analyzer 122 in a location accessible to the analyzer. As disclosed above, the breathing data (e.g., the nasal bridge vibration data 112) generated by the sensor(s) 106 as the user 104 breathes is transmitted to the breathing pattern analyzer 122. In the illustrated example, the database 200 provides means for storing the breathing data. In some examples, the database 200 stores the breathing data 112 over time to generate historical breathing data (e.g., historical nasal vibration data 112).

The example breathing pattern analyzer 122 includes an analog-to-digital (A/D) converter 202. In the illustrated example, the A/D converter 202 provides means for sampling the raw analog nasal bridge vibration signal data 112 at a particular sampling rate and converting the analog data to digital signal data for analysis by the example breathing pattern analyzer 122.

The example breathing pattern analyzer 122 of FIG. 2 includes a filter 204 (e.g., a band pass filter). In the illustrated example, the filter 204 provides means for filtering the nasal bridge vibration data 112. Breathing data such as the nasal bridge vibration data 112 may be located in different frequency bands based on whether the user is breathing orally or nasally. For example, the nasal vibration data 112 associated with nasal breathing can include frequencies within the range of 3-4 kHz while nasal bridge vibration data associated with oral (e.g., mouth) breathing can include frequencies within the range of 150-300 Hz. The example filter 204 of FIG. 2 filters the nasal bridge vibration data 112 to pass frequencies within a known frequency band associated with nasal breathing (e.g., 3-4 kHz). In some examples, the filter 204 includes a high-pass filter to filter the vibration data with respect to the higher frequencies associated with nasal breathing and a low-pass filter to filter the vibration with respect to the lower frequencies associated with oral breathing (e.g., 150-300 Hz). In some examples, the digital nasal vibration data 112 is separately provided to the low pass filter and the high pass filter and the outputs of the filters 204 are combined by, for example, a summer. In some examples, the breathing data include data from one or more sensor(s), in addition to the nasal sensor(s). For example, sound data may be collected to identify mouth breathing data. In some such examples, this additional breathing data is digitalized by the A/D/converter 202 (if necessary), filtered by the filter(s) 204 (if necessary), and may be combined with the filtered nasal bridge vibration 112 (e.g., via a summer, an aggregator, etc.) to generate filtered breathing data. In other examples, the other data is not collected and/or not combined with the nasal bridge vibration data and, thus, processed separately. The following discussion applies to all approaches (e.g., nasal vibration data only, nasal vibration data processed separately from additional breathing data, and nasal vibration data and other vibration data combined/aggregated and subsequently processed/analyzed together). The filtered breathing data can be stored in the database 200.

The example breathing pattern analyzer 122 of FIG. 2 includes a signal envelope calculator 206. In the illustrated example, the signal envelope calculator 206 provides means for calculating an envelope (e.g., a root-mean-square (RMS) envelope) for the filtered breathing data which may or may not be only the nasal bridge vibration signal data 112. The RMS envelope profile 208 for the breathing data indicates changes in the breathing data (e.g., the nasal bridge vibration data 112) over time, such as changes in amplitude. In the example of FIG. 2, the changes in the breathing data represent changes in breathing energy over the respective phases of one or more breathing cycles (e.g., inspiration, inspiration pause, expiration, and expiration pause). In the example of FIG. 2, the envelope profile 208 is stored in the database 200.

The example breathing pattern analyzer 122 of FIG. 2 includes a breathing phase detector 210. In the illustrated example, the example breathing phase detector 210 provides means for analyzing the envelope profile 208 generated by the signal envelope calculator 206 and identifies breathing phases 212 for each breathing cycle based on one or more features of the envelope profile 208. For example, the breathing phase detector 210 analyzes features or characteristics of the data in the envelope profile 208 over one or more time windows (e.g., amplitude changes, changes in slope).

For example, the breathing phase detector 210 identifies amplitude levels of the data in the envelope profile 208. In some examples the amplitude levels correspond to peaks (e.g., inflection points) in the envelope profile 208. In some examples, the breathing phase detector 210 identifies distances between peaks in the envelope profile 208. The breathing phase detector 210 can identify other features of the envelope profile 208, such as a frequency of the peaks, a slope of the peaks, width of the peaks, amplitude variations other than those related to the peaks, etc.

The example breathing phase detector 210 of FIG. 2 identifies the breathing phases 212 of a breathing cycle. In some examples, the breathing phase detector 210 identifies one or more of the inspiration phase, the inspiration pause, the expiration phase, and the expiration pause, by comparing the amplitude of the peaks to breathing phase thresholds 214 for a corresponding one of the breathing phases. The breathing phase thresholds 214 can be defined by one or more user inputs and/or historical data for previously analyzed envelope profile(s) 208. The breathing phase thresholds 214 can include, for example, reference amplitude values corresponding to the inspiration phase, the inspiration pause, the expiration phase, and/or the expiration pause. In some examples, the breathing phase thresholds 214 include average distances or durations between peaks corresponding to, for example, different inspiration phases. In some examples, the breathing phase thresholds 214 include reference data for other features of the envelope profile 208, such as breathing cycle frequency.

The reference threshold data can be based on data previously collected for the user (e.g., the user 104) and/or for other users. In some examples, the breathing phase thresholds 214 are defined based on one or more user characteristics, such as age, health condition, etc. In some examples, the breathing phase thresholds 214 are defined based on one or more user activities such as running, walking, sitting, etc. In some examples, the breathing phase thresholds 214 are defined during calibration of the breathing pattern analyzer 122 for the user 104. In the example of FIG. 2, the breathing phase thresholds 214 are stored in the database 200.

In the example of FIG. 2, the breathing phase detector 210 identifies the breathing phases 212 (i.e., the inspiration phase, the inspiration pause, the expiration phase, and the expiration pause) by comparing the amplitude levels of the envelope profile 208 to the breathing phase thresholds 214. For example, if the amplitude of a peak in the envelope profile 208 satisfies or is within a threshold range (e.g., +/−) of a reference threshold amplitude for the inspiration phase, then the breathing phase detector 210 identifies the data of the peak as corresponding to the inspiration phase. In some examples, the breathing phase detector 210 identifies the breathing phase 212 based on change(s) in the data, such as an increase in amplitude over time as associated with the inspiration phase (e.g., based on the breathing phase thresholds 214).

As another example, the envelope profile 208 can include data having decreased amplitude relative to the data identified as associated with the inspiration phase. The breathing phase detector 210 compares the data with the decreased amplitude to the breathing phase thresholds 214 to identify the data as associated with, for example, the expiration phase. As disclosed herein, the example breathing phase detector 210 can analyze other characteristics of the signal data in addition to or in alternative to the peak amplitudes, such as changes in slope, changes in amplitudes other than the peaks, etc. Thus, the example breathing phase detector 210 analyzes the envelope profile 208 to extract data features corresponding to the breathing phases 212 of one or more breathing cycles and to classify the data as associated with certain phases (e.g., the inspiration phase, the inspiration pause, the expiration phase, the expiration pause). The breathing phase detector 210 can identify the breathing phases 212 for all of the data of the envelope profile 208 and/or or portions thereof based on user input(s).

The example breathing pattern analyzer 122 of FIG. 2 includes a phase timing calculator 216. In the illustrated example, the phase timing calculator 216 provides means for determining the start time and the end time of each of the breathing phases 212 identified by the breathing phase detector 210. Based on the start and end times, the phase timing calculator 216 calculates a breathing phase time period 218 for each breathing phase 212. The breathing phase time periods 218 include respective durations of corresponding ones of the breathing phases.

For example, the phase timing calculator 216 correlates the data identified as corresponding to the inspiration phase by the breathing phase detector 210 with time. Using the time-based correlation, the phase timing calculator 216 identifies the start time of the inspiration phase and the end time of the inspiration phase and calculates an inspiration phase time period Tᵢ (e.g., a duration of the inspiration phase, or an amount of time for the user 104 to take oxygen into his or her lungs). Similarly, the phase timing calculator 216 calculates the inspiration pause time period Tᵢₚ, the expiration phase time period Tₑ, and the expiration phase Tₑₚ. The values of the breathing phase time periods Tᵢ, Tᵢₚ, Tₑ, Teₑₚ indicate a total duration of a breathing cycle. In the example of FIG. 2, the breathing phase time periods 218 (e.g., Tᵢ, Tᵢₚ, Tₑ, Tₑₚ) are stored in the database 200.

In some examples, for a given envelope profile 208, the breathing phase detector 210 is unable to detect the breathing phase(s) 212 relative to the breathing phase threshold(s) 214. In such examples, the phase timing calculator 216 is unable to determine the breathing phase time period(s) 218 for all or a portion of the envelope profile(s) 208. For example, a portion of an envelope profile 208 may include a substantial reduction in peak amplitude over a period of time. In such examples, the breathing phase detector 210 may determine that the user is breathing orally (e.g., through his or her mouth) rather than nasally and/or has switched between breathing nasally and breathing orally. As disclosed herein, the breathing pattern analyzer 122 flags such data to generate an alert.

The example breathing pattern analyzer 122 of FIG. 2 includes a breathing pattern detector 220. In the illustrated example, the example breathing pattern detector 220 provides means for generating one or more breathing pattern metrics 222 based on the breathing phase time periods 218. In the example of FIG. 2, the breathing phase time period(s) 218 and the breathing pattern metric(s) 222 are indicative of the user's breathing pattern and are used to associate the breathing pattern with an activity such as smelling.

For example, the breathing pattern detector 220 can generate a first breathing metric 222 based on the inspiration phase time period Tᵢ and the expiration phase time period Tₑ using the following equation:

$${f}_{R} = \frac{1}{{T}_{i} + {T}_{ip} + {T}_{e} + {T}_{ep}} ,$$

The first example metric 222 defines a ratio that expresses a difference between a duration of the inspiration phase and a duration of the expiration phase.

The breathing pattern detector 220 can generate a second breathing metric 222 using the following equation:

$$\frac{{T}_{i}}{{T}_{e}} . \qquad ( Example Metric 1 )$$

where f_{R} is a respiration frequency, Tᵢₚ is the inspiration pause time period, and Tₑₚ is the expiration pause time period (Example Metric 2).

The second example metric 222 defines a respiration frequency f_{R} for a breathing cycle. In some examples, the breathing pattern detector 220 calculates a variance of the respiration frequency f_{R} across two or more breathing cycles.

The example breathing pattern detector 220 can use the respiration frequency f_{R} to generate one or more metrics 222 that determine a duration of a breathing phase relative to the total duration of the breathing cycle. For example, the breathing pattern detector 220 can calculate the following breathing pattern metrics 222 for each breathing phase:

Tᵢ*f_{R} (Example Metric 3);

Tᵢₚ*f_{R} (Example Metric 4);

Tₑ*f_{R} (Example Metric 5);

Tₑₚ*f_{R} (Example Metric 6).

For example, the metric Tᵢₚ*f_{R} represents a duration of the inspiration pause relative to the total duration of the breathing cycle and the metric Tₑ*f_{R} represents a duration of the expiration pause relative to the total duration of the breathing cycle.

As an example, during quiet (or idle) breathing, the inspiration phase time period Tᵢ can have a value of 2.58 seconds, the inspiration pause time period Tᵢₚ can have a value of 0.23 seconds, the expiration phase time period Tₑ can have a value of 1.72 seconds, and the expiration pause time period Tₑₚ can have a value of 2.13 seconds. Thus, the total quiet (or idle) breathing cycle has a duration of 6.66 seconds. Based on the first example metric, above, the ratio of the duration of the inspiration phase to the duration of the expiration phase is 1.5 (e.g., 2.58/1.72). The respiration frequency f_{R is} 0.15 (e.g., 1/(2.58+0.23+1.72+2.13). The respective durations of the breathing phases relative to the total duration of the breathing cycle are approximately 0.6 for the inspiration phase (e.g., 2.58*0.15), 0.03 for the inspiration pause (e.g., 0.23*0.15), 0.2 for the expiration phase (e.g., 1.72*0.15), and 0.3 for the expiration pause.

In some examples, the breathing pattern detector 220 generates the breathing pattern metric(s) 222 in substantially real-time as, for example, the vibration data 112 is receive by the breathing pattern analyzer 122 of FIG. 2 and processed (e.g., by the filter 204, the signal envelope calculator 206). In some examples, the breathing pattern detector 220 generates the breathing pattern metric(s) 222 for a particular portion of the vibration data 112 received by the breathing pattern analyzer 122. For instance, in some examples, the breathing pattern detector 220 calculates the breathing pattern metric(s) 222 if a change in a respective duration of any of the breathing phases 212 is detected. In some examples, the breathing pattern detector 220 selectively generates the breathing pattern metric(s) 222 for one or more breathing phases 212. For example, the breathing phase detector 210 can determine the duration of the inspiration pause relative to the total breathing cycle (e.g., example metric 4, above) when (e.g., only when) the inspiration pause time period (i.e., Tᵢₚ) exceeds a threshold value. Thus, in some examples, the breathing pattern detector 220 intelligently determines which metric(s) 222 to generate based on breathing phase(s) 212 and/or the breathing phase time period(s) 218. In other examples, the breathing pattern detector 220 generates the breathing pattern metric(s) for all of the vibration data 112 received by the breathing pattern analyzer 122.

In the example of FIG. 2, the breathing phase time period(s) 218 and/or the breathing pattern metric(s) 222 represent a breathing pattern for the user. The durations of the breathing phases 212 and features such as the respiration frequency f_{R}, the ratio of the inspiration phase duration to the expiration phase duration, and/or the durations of the respective breathing phases 212 relative to the total breathing cycle duration, define characteristics of the user's breathing and thus, define the user's breathing pattern at a given time. The breathing pattern detector 220 can generate other metrics than the example metrics mentioned above. The breathing pattern metric(s) 222 can be defined by one or more user inputs. In the example of FIG. 2, the breathing pattern metric(s) 222 are stored in the database 200.

The example breathing pattern analyzer 122 of FIG. 2 includes a breathing activity detector 224. In the illustrated example, the example breathing activity detector 224 provides means for analyzing the breathing pattern metrics to identify the user's breathing pattern and to associate the breathing pattern with one or more activities, such as breathing during exercise, quiet breathing, smelling, etc. The example breathing activity detector 224 applies one or more breathing pattern rule(s) 226 to associate the breathing pattern with the one or more activities.

For example, with respect to the first example breathing pattern metric defining the ratio of the durations of the inspiration phase and the expiration phase (e.g., Tᵢ/Tₑ), an example breathing pattern rule 226 can indicate that during quiet breathing, the duration of the inspiration phase and the expiration phase are substantially equal (e.g., a ratio of 1). Another example breathing pattern rule 226 can indicate that when the user smells an odor, the duration of the inspiration phase is expected to be longer than the expiration phase.

Other breathing pattern rule(s) 226 relate to the respiration frequency f_{R}. For example, a breathing pattern rule 226 can indicate that the respiration frequency f_{R} decreases when the user is engaged in quiet breathing as compared to when the user is breathing during exercise. Another example breathing pattern rule 226 can indicate that a variance of the respiration frequency f_{R} increases during smelling.

Other breathing pattern rule(s) 226 relate to the ratios that measure a duration of each breathing phase relative to the total duration of the breathing cycle (e.g., Tₑₚ*f_{R}). For example, a breathing pattern rule 226 can indicate that during smelling, the respective durations of the inspiration pause and the expiration pause relative to the total duration of the breathing cycle are reduced as compared to when the user is engaged in quiet breathing.

The breathing pattern rule(s) 226 can include other rules such as average total durations of breathing cycles for different activities and/or rules customized based on user characteristics such as age, gender, health condition, etc. In the example of FIG. 2, the breathing pattern rule(s) 226 are defined by one or more user inputs and/or historical data for previously analyzed envelope profile(s). In the example of FIG. 2, the breathing pattern rule(s) are stored in the database 200.

The example breathing activity detector 224 evaluates the breathing pattern metric(s) 222 based on the breathing pattern rule(s) 226 to identify the breathing activities associated with the breathing patterns, such as smelling, quiet breathing, etc. In the example of FIG. 2, the example breathing activity detector 224 uses the breathing pattern metric(s) 222 to differentiate between different breathing activities. In some examples, the breathing activity detector 224 applies the rule(s) 226 to the breathing pattern metric(s) 222 generated for two or more breathing cycles over time. In some such examples, the breathing activity detector 224 determines that breathing data (e.g., the vibration data 112, the corresponding signal envelope profile data) is indicative of a particular activity (e.g., smelling) if the breathing activity detector 224 determines that the two or more breathing cycles are associated with the same activity. In other examples, the breathing activity detector 224 determines there is a change in the breathing activity based on changes (e.g., differences) in the metric(s) 222 between two or more breathing cycles.

Based on the analysis of the breathing pattern metric(s) 222 and the application of the breathing pattern rule(s) 226, the breathing activity detector 224 of FIG. 2 generates one or more breathing activity classifications 228. The breathing activity classification(s) 228 indicate that the breathing data for one or more breathing cycles is associated with an activity such as smelling, quiet breathing, breathing during exercise, etc. In the example of FIG. 2, the breathing activity classification(s) 228 are stored in the database 200.

Thus, the example breathing activity detector 224 identifies one or more breathing activities corresponding to the user's breathing patterns over time (e.g., smelling, quiet breathing, etc.) based on the breathing pattern metric(s) 222 and the breathing pattern rule(s) 226. In other examples, the example breathing pattern analyzer 122 of FIG. 2 automatically classifies the breathing activities associated with the breathing data based on the breathing phase time period(s) 218 (e.g., Tᵢ, Tᵢₚ, Tₑ, Tₑₚ) and one or more machine learning algorithms. In some examples, the example breathing pattern analyzer 122 includes a classifier 230 that receives the breathing phase time period(s) 218 as input(s) and provides means for automatically identifying the breathing activity based on the breathing phase time period(s) 218. In the example of FIG. 2, the classifier 230 is trained to recognize the breathing activity based on supervised or unsupervised machine learning algorithms (e.g., artificial neural networks).

For example, the breathing pattern analyzer 122 of FIG. 2 includes a trainer 232 including means for training the classifier 230. In some examples, the trainer 232 trains the classifier 230 based on the breathing phase(s) 212, the breathing phase time period(s) 218, the breathing pattern metric(s) 222, and/or the breathing activity classification(s) 228 determined for previously collected vibration data 112 (e.g., historical data, sample data). For example, the breathing phase(s) 212 identified by the breathing phase detector 210, the breathing phase time period(s) 218 calculated by the phase timing calculator 216, the breathing pattern metric(s) 222 generated by the breathing pattern detector 220, and/or the breathing activity classification(s) 228 generated by the breathing activity detector 224 can serve as inputs that are used by the trainer 232 to train the classifier 230. In some examples, the trainer associates the breathing phase(s) 212, the breathing phase time period(s) 218, the breathing pattern metric(s) 222, and/or the breathing activity classification(s) 228 with the envelope profile(s) 208 from which the phases, time periods, metrics, and/or classifications were derived to train the trainer 232 to recognize features of envelope profile(s) 208 corresponding to certain breathing activities. In other examples, the trainer 232 uses the raw envelope profile(s) 208 to train the classifier 230 based on, for example, amplitudes of peaks in the envelope profile(s) 208.

In the example of FIG. 2, when the classifier 230 receives breathing phase time period(s) 218 (e.g., Tᵢ, Tᵢₚ, Tₑ, Tₑₚ) for one or more breathing cycles in the vibration data 112 that have not previously been analyzed, the classifier 230 assigns the breathing activity classification(s) 228 (e.g., quiet breathing, smelling) to the breathing data based on the training. Thus, in some examples, the breathing pattern analyzer 122 of FIG. 2 automatically determines the breathing activities based on the breathing phase time period(s) 218 and without, for example, generating the breathing pattern metric(s) 222 as part of a machine learning-based analysis.

In examples where the breathing activity classification(s) 228 are assigned by the classifier 230 of FIG. 2 based on machine learning algorithm(s), the breathing pattern analyzer 122 can verify the classification(s) 228 generated by the classifier 230. For example, the breathing pattern analyzer 122 of FIG. 2 includes a post-processing engine 234. The post-processing engine 234 provides means for evaluating or post-processing the breathing activity classification(s) 228 generated by the classifier 230 to check for any error(s) in the classification(s) 228 and means for correcting the error(s) (e.g., by updating the breathing activity classification(s) 228 with the correct classification(s)). In some examples, the post-processing engine 234 determines if the classifier 230 should be re-trained. In the example of FIG. 2, the trainer 232 uses any corrections to the breathing activity classification(s) 228 during post-processing to train (or re-train) the classifier 230 to identify the breathing activities.

For instance, the post-processing engine 234 of this example evaluates the breathing activity classification(s) 228 assigned by the classifier 230 with respect to consistency of the classification(s) 228 across breathing cycles. The post-processing engine 234 applies one or more verification rules 236 to evaluate the classification(s) 228. For example, for three adjacent breathing cycles each including an inspiration phase time period Tᵢ meeting a threshold, the post-processing engine 234 verifies that the classifier 230 assigned the same breathing activity classification 228 to each of the breathing cycles (e.g., quiet breathing). The post-processing engine 234 verifies that the classifier 230 did not identify one of the breathing cycles as associated with a different breathing activity classification 228 (e.g., a breathing activity classification associated with vigorous exercise for a breathing cycle located between two breathing cycles classified as quiet breathing). The verification rule(s) 236 can include known values of breathing phase time periods associated with different breathing activities, error thresholds for the classifier 230 to trigger re-training of the classifier 230, etc. In some examples, the verification rule(s) 236 are based on the breathing pattern rule(s) 226 and/or other user inputs. In the example of FIG. 2, the verification rule(s) 236 are stored in the database 200.

The breathing pattern analyzer 122 of this example includes an alert generator 238. In the illustrated example, the alert generator 238 provides means for determining whether one or more alerts 240 should be generated and means for activating one or more output devices to generate the alert(s) 240. For example, based on the breathing activity classification(s) 228 and/or the breathing pattern metric(s) 222, the alert generator 238 determines the alert(s) 240 to be generated by the breathing pattern analyzer 122 and activates the output device(s) (e.g., the wearable device 102, the wearable or non-wearable user device 118, another device in communication with the cloud-based device 120) to generate the alert(s) 240. In some examples, the alert generator 238 determines that the alert(s) 240 should be generated if one or more conditions are met. For example, the alert generator 238 may determine that the alert(s) 240 should be generated if a change in the breathing activity is identified by the breathing activity detector 224 relative to a previously identified activity. As another example, the alert generator 238 may determine that the alert(s) 240 should be output if the breathing phase time period(s) 218 and/or the breathing pattern metric(s) 222 meet certain threshold(s) (e.g., as defined by user input(s)). In some examples, the condition(s) are based on user characteristics such as age, gender, fitness level, etc. The alert(s) 240 can include visual alerts, audio alerts, tactical alerts, etc. The alert(s) 240 may be presented to the user and/or transmitted to a third party such as a physician, a caregiver, etc. In some examples, the format and/or content of the alert(s) 240 are customized based on user setting(s), the type of output device (e.g., whether the output device includes a display screen), and/or user characteristic(s) (e.g., age).

For example, the breathing activity detector 224 may determine that the user is engaged in quiet breathing based on the vibration data 112 analyzed by the breathing pattern analyzer 122 in substantially real-time. During the substantially real-time analysis, the breathing activity detector 224 may determine that the breathing pattern metric(s) 222 have surpassed a threshold associated with quiet breathing and are closer to a threshold associated with breathing during exercise (e.g., based on the breathing pattern rule(s) 226). In such examples, the alert generator 238 determines that an alert 240 instructing the user to alter his or her activity and/or breathing to return to quiet breathing (e.g., to take deeper breaths) should be generated. The alert generator 240 can activate the user device 118 to display a visual alert 240 via a graphical user interface, such as a statement recommending that the user adjust (e.g., slow) his or her activity and/or breathing rate. In other examples, the alert 240 can include a vibration or a sound executed at the wearable device 102 to remind the user to slow down his or her activity and/or breathing.

In some examples, the alert(s) 240 include information about the user's breathing pattern over time during an activity such as running. For example, the alert(s) 240 can include data about the user's respiration frequency, the duration of the inspiration period, the duration of the expiration pause, etc. Such data can be presented to a user such as an athlete as breathing performance data that is monitored as part of a fitness regime. In such examples, the alert(s) 240 can be generated in substantially real-time (e.g., when the breathing pattern analyzer 122 analyzes the vibration data in substantially real-time) or at later time (e.g., if the vibration data 112 is received by the breathing pattern analyzer after the activity is complete).

In some examples, the alert generator 238 determines that the alert(s) 240 should be generated if the breathing phase detector 210 detects a shift from nasal breathing to oral breathing. For example, the breathing activity detector 224 may determine that the user is engaged in quiet breathing based on one or more breathing cycles during a first time period. During a second time period, the breathing phase detector 210 may determine that the user has shifted to oral breathing (e.g., based on reduced frequency or amplitude of the vibration data 112). In such examples, the alert generator 238 may activate the output device(s) to generate alert(s) 240 based on the detected shift from nasal breathing to oral breathing. The alert(s) 240 can flag potential health problems if, for example, the user is breathing orally during quiet breathing and/or provide recommendations for more efficient breathing during everyday activities such as sitting at a desk.

As another example, the alert generator 238 determines that the alert(s) 240 should be generated based on the amplitude(s) of the signal data corresponding to the inspiration phase(s) and the expiration phase(s). Amplitude(s) associated with inspiration phase data and/or the expiration phase data during, for example, quiet breathing can be indicative of a shallowness of the user's breathing. If the breathing activity detector 224 determines that the amplitude(s) are below predefined threshold values for non-shallow breathing, the alert generator 238 activates the output device(s) to generate the alert(s) 240 indicating potential health problems related to lung capacity and/or muscular problems that are preventing the user from breathing efficiently during quiet breathing.

As another example, the breathing activity detector 224 may determine that duration(s) of time between the inspiration pause(s) and the expiration pause(s) exceed a predefined threshold or are below the threshold (e.g., during quiet breathing). In such examples, the alert generator 238 activates the output device(s) to generate the alert(s) 240 to flag potential anomalies in the user's breathing.

In some examples, the breathing pattern metric(s) 222 are analyzed with other user data such as age, weight, height, body mass index (BMI), gender, etc. The user data can include known (e.g., average) user data for one or more users and/or known (e.g., calibration) data for the user whose breathing data is being analyzed. In such examples, the alert generator 238 can activate the output device(s) to generate the alert(s) 240 if the analysis of the breathing pattern metric(s) 222 in view of the user data indicates that, for example, the user's quiet breathing cycle frequency is above or below an average breathing cycle for other users have similar characteristics such as age or gender. The alert generator 238 can activate the output device(s) to generate the alert(s) 240 based on the comparisons to known user data during different activities (e.g., light exercise, vigorous exercise) to inform the user as to whether their breathing pattern metrics are potentially abnormal relative to other users during the activities.

The example breathing pattern analyzer 122 of FIG. 2 includes a communicator 242. The communicator 242 includes means for communicating with one or more output devices, which can include the wearable device 102 and/or the user device 118, to instruct and/or activate the output device(s) to generate the alert(s) 240 for presentation via display(s) of the output device(s), speaker(s) of the output device(s), etc.

While an example manner of implementing the example breathing pattern analyzer 122 is illustrated in FIGS. 1 and 2, one or more of the elements, processes and/or devices illustrated in FIGS. 1 and 2 may be combined, divided, re-arranged, omitted, eliminated and/or implemented in any other way. Further, the example database 200, the example A/D converter 202, the example filter 204, the example signal envelope calculator 206, the example breathing phase detector 210, phase timing calculator 210, the example breathing pattern detector 220, the example breathing activity detector 224, the example classifier 230, the example trainer 232, the example post-processing engine 234, the example alert generator 238, the example communicator 242 and/or, more generally, the example breathing pattern analyzer 122 of FIGS. 1 and 2 may be implemented by hardware, software, firmware and/or any combination of hardware, software and/or firmware. Thus, for example, any of the example database 200, the example A/D converter 202, the example filter 204, the example signal envelope calculator 206, the example breathing phase detector 210, phase timing calculator 210, the example breathing pattern detector 220, the example breathing activity detector 224, the example classifier 230, the example trainer 232, the example post-processing engine 234, the example alert generator 238, the example communicator 242 and/or, more generally, the example breathing pattern analyzer 122 of FIGS. 1 and 2 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example database 200, the example A/D converter 202, the example filter 204, the example signal envelope calculator 206, the example breathing phase detector 210, phase timing calculator 210, the example breathing pattern detector 220, the example breathing activity detector 224, the example classifier 230, the example trainer 232, the example post-processing engine 234, the example alert generator 238, the example communicator 242 and/or, more generally, the example breathing pattern analyzer 122 of FIGS. 1 and 2 is/are hereby expressly defined to include a non-transitory computer readable storage device or storage disk such as a memory, a digital versatile disk (DVD), a compact disk (CD), a Blu-ray disk, etc. including the software and/or firmware. Further still, the example breathing pattern analyzer of FIGS. 1 and 2 may include one or more elements, processes and/or devices in addition to, or instead of, those illustrated in FIGS. 1 and 2, and/or may include more than one of any or all of the illustrated elements, processes and devices.

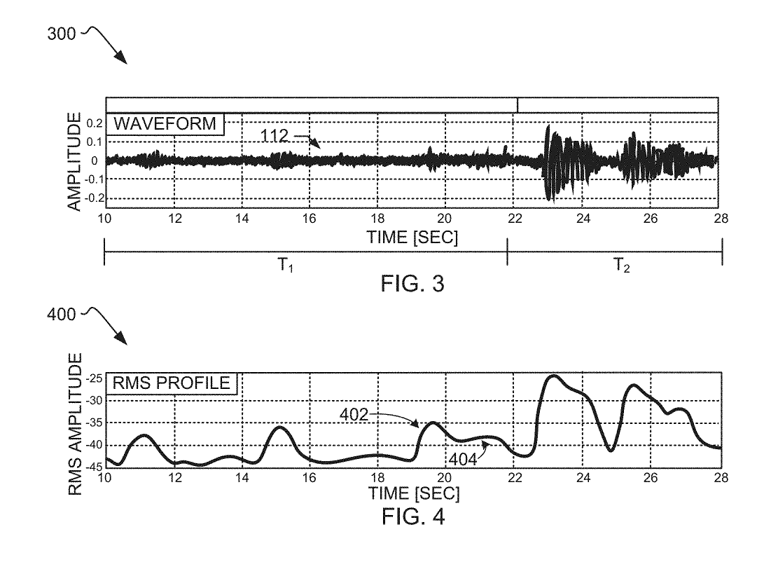

FIG. 3 illustrates a graph 300 including example nasal bridge vibration data 112 collected from a user (e.g., the user 104) wearing the example wearable device 102 of FIG. 1. In the example of FIG. 3, the nasal bridge vibration data 112 is generated by the sensor(s) 106 (e.g., the piezoelectric sensor(s)) of the wearable device 102. In the example of FIG. 3, the nasal bridge vibration data 112 was collected during a first time period t₁ when the user 104 was engaged in a first breathing activity (e.g., quiet breathing) and a second time period t₂ when the user 104 was engaged in a second breathing activity (e.g., smelling a flower). As illustrated in FIG. 3, amplitudes values of the nasal bridge vibration data 112 vary with respect to the different activities. As disclosed herein, the example breathing pattern analyzer 122 of FIGS. 1 and 2 uses the differences in the features of the nasal bridge vibration data 112 to identify the breathing phase(s) 212 and corresponding breathing patterns for different breathing activities.

FIG. 4 illustrates a root-mean-square (RMS) envelope profile 400 of the example nasal bridge vibration data 112 of FIG. 3. The RMS envelope profile 400 of FIG. 4 can be generated by the signal envelope calculator 206 of the example breathing pattern analyzer 122 of FIG. 2. As shown in FIG. 4, the example RMS envelope profile 400 includes data having different amplitudes, durations, etc. As disclosed herein, the example breathing pattern analyzer 122 analyzes the different features of the RMS envelope profile 400 to determine the breathing pattern(s) (e.g., based on the breathing phase time period(s) 218, the breathing pattern metric(s) 222) and identify the breathing activity.

For example, based on differences in amplitude in the data of the RMS envelope profile 400, the breathing phase detector 210 of the example breathing pattern analyzer 122 of FIG. 2 identifies a first data portion 402 and a second data portion 404. Based on the breathing phase threshold(s) 214, the example breathing phase detector 210 identifies the respective breathing phases 212 for the first data portion 402 (e.g., inspiration phase) and the second data portion 404 (e.g., expiration phase). The phase timing calculator 216 calculates the breathing phase time periods 218 for the first data portion 402 (e.g., the inspiration phase time period Tᵢ) and the second data portion 404 (e.g., the expiration phase time period Tₑ). Based on the breathing phase time periods 218, the breathing pattern analyzer 122 determines the breathing activity associated with the first and second data portion 402, 404. For example, the classifier 230 can generate the breathing activity classification 228 using machine learning algorithm(s) or the breathing activity detector 224 can generate the breathing activity classification 228 based on the metric(s) 222 calculated by the breathing pattern detector 220 and the breathing pattern rule(s) 226.

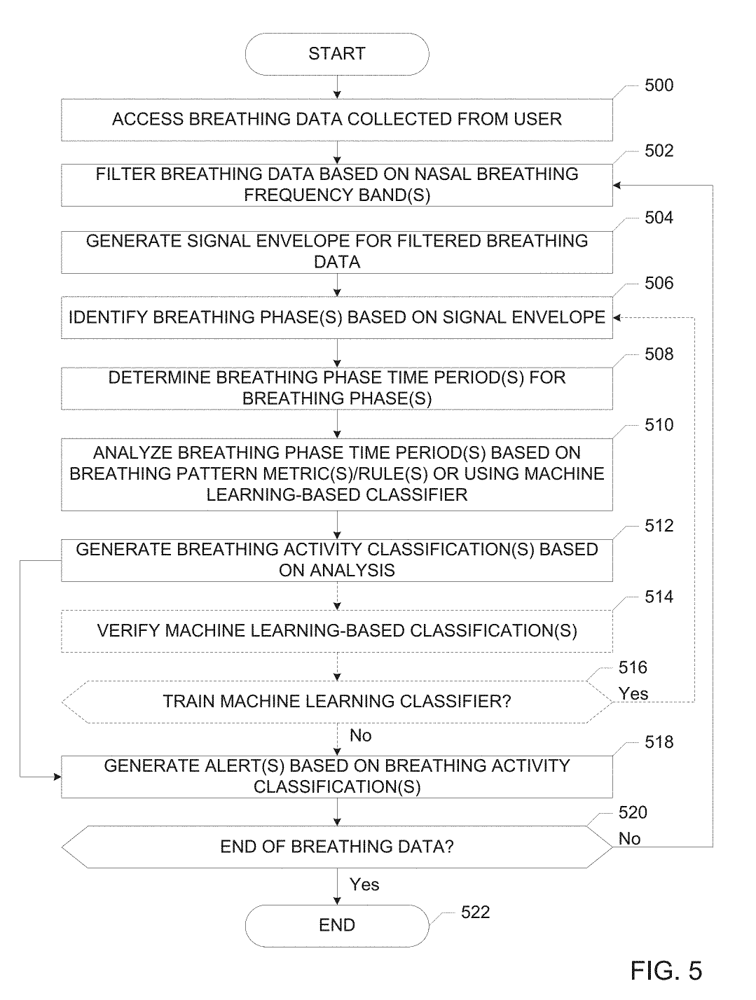

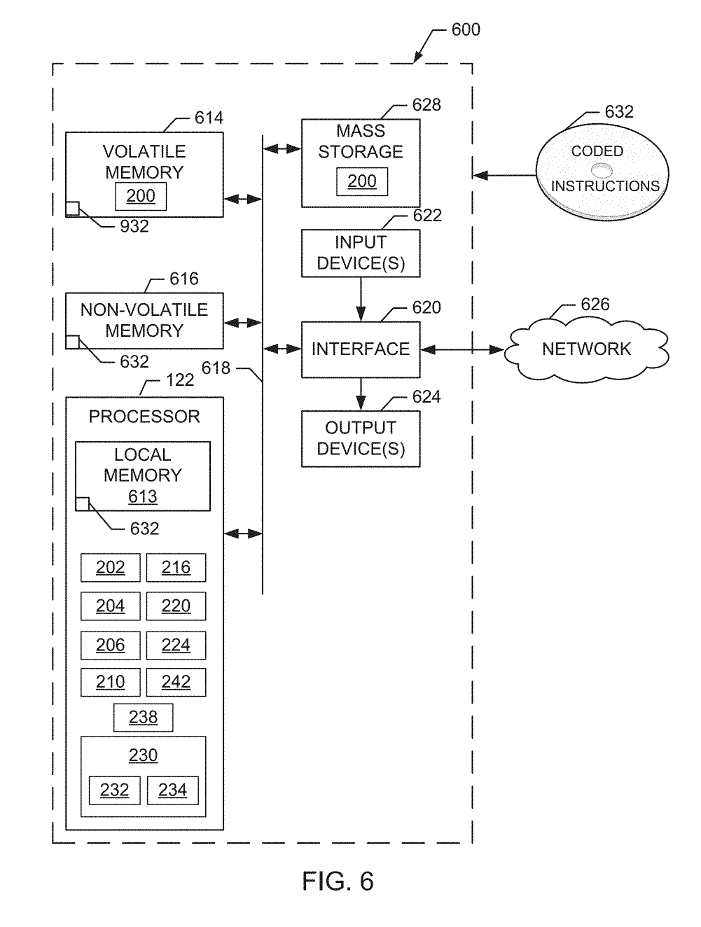

A flowchart representative of example machine readable instructions for implementing the example system 100 of FIGS. 1 and 2 is shown in FIG. 5. In this example, the machine readable instructions comprise a program for execution by a processor such as the processor 122 shown in the example processor platform 600 discussed below in connection with FIG. 6. The program may be embodied in software stored on a non-transitory computer readable storage medium such as a CD-ROM, a floppy disk, a hard drive, a digital versatile disk (DVD), a Blu-ray disk, or a memory associated with the processor 122, but the entire program and/or parts thereof could alternatively be executed by a device other than the processor 122 and/or embodied in firmware or dedicated hardware. Further, although the example program is described with reference to the flowchart illustrated in FIG. 5, many other methods of implementing the example system 100 and/or components thereof may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, a Field Programmable Gate Array (FPGA), an Application Specific Integrated circuit (ASIC), a comparator, an operational-amplifier (op-amp), a logic circuit, etc.) structured to perform the corresponding operation without executing software or firmware.

As mentioned above, the example processes of FIG. 5 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. “Including” and “comprising” (and all forms and tenses thereof) are used herein to be open ended terms. Thus, whenever a claim lists anything following any form of “include” or “comprise” (e.g., comprises, includes, comprising, including, etc.), it is to be understood that additional elements, terms, etc. may be present without falling outside the scope of the corresponding claim. As used herein, when the phrase “at least” is used as the transition term in a preamble of a claim, it is open-ended in the same manner as the term “comprising” and “including” are open ended.

FIG. 5 is a flowchart of example machine readable-instructions that, when executed, cause the example breathing pattern analyzer 122 of FIGS. 1 and/or 2 to identify breathing activities based on breathing data (e.g., nasal bridge vibration data 112) collected from a user (e.g., the user 104 of FIG. 1). In the example of FIG. 5, the breathing data can be collected via sensor(s) 106 of the wearable device 102 of FIG. 1. The example instructions of FIG. 5 can executed by the breathing pattern analyzer 122 of FIGS. 1 and/or 2. The breathing pattern analyzer 122 of FIGS. 1 and/or 2 can be located at the processor 114 of the wearable device 102, the processor 116 of the user device 118, and/or the cloud-based device 120. The instructions of FIG. 5 can be executed in substantially real-time as the breathing data is generated and received by the breathing pattern analyzer 122 or at some time after the breathing data is generated.

The example filter 204 of the breathing pattern analyzer 122 of FIG. 2 accesses breathing data collected from a user (block 500). For example, the breathing pattern analyzer 122 accesses the nasal bridge vibration data 112 generated over time from the user 104 wearing the wearable device 102 including the piezoelectric sensor(s) 106 (block 500). In some examples, the nasal bridge vibration 112 is converted by the A/D converter 202 of the breathing pattern analyzer 122 to digital signal data. In other examples, the nasal bridge vibration data 112 is received by the breathing pattern analyzer 122 as digital signal data (e.g., after being converted by another processor).

The example filter 204 filters the breathing data (e.g., the nasal bridge vibration data 112) with respect to frequency bands associated with nasal breathing (e.g., 3-4 kHz) and to remove data at frequencies associated with oral breathing (e.g., 150-300 Hz) (block 502).

The example signal envelope calculator 206 generates a signal envelope profile 208, 400 for the breathing data (block 504). For example, the signal envelope calculator 206 calculates a root-mean-square envelope for the nasal bridge vibration data 112. The signal envelope profile 208, 400 represents changes in breathing energy over one or more breathing cycles (i.e., inspiration, inspiration pause, expiration, expiration pause) as represented by, for example, amplitude changes in the signal envelope.

The example breathing phase detector 210 identifies breathing phase(s) 212 based on the changes in the signal envelope profile 208, 400 (block 506). For example, the breathing phase detector 210 identifies changes in amplitudes of peaks in the data of the signal envelope profile 208, 400 (e.g., the data portions 402, 404 of the signal envelope profile 400 of FIG. 4). The breathing phase detector 210 compares the peak amplitudes to predefined breathing phase thresholds 214 to identify the inspiration phase, the inspiration pause, the expiration phase, and the expiration pause for one or more breathing cycles represented by the signal envelope profile 208, 400. Additionally or alternatively, the breathing phase detector 210 can identify the breathing phases based on, for example, changes in slope in the signal data, amplitude changes in the signal data other than those related to the peaks, etc.

The breathing phase timing calculator 216 determines the breathing phase time period(s) 218 corresponding to each of the breathing phase(s) 212 identified by the breathing phase detector 210 (block 508). For example, the breathing phase timing calculator 216 identifies the start and end times of each breathing phase 212 based on the signal envelope profile 208, 400. The breathing phase timing calculator 216 calculates the inspiration phase time period Tᵢ, the inspiration pause time period Tᵢₚ, the expiration phase time period Tₑ, and the expiration pause time period Tₑₚ based on the start and end times to determine a duration of each breathing phase.

The example instructions of FIG. 5 include analyzing the breathing phase time period(s) 218 (e.g., Tᵢ, Tᵢₚ, TₑTₑₚ) to identify breathing activities being performed by the user (block 510). In some examples, the breathing phase time period(s) 218 are analyzed based on one or more breathing pattern metrics 222. In such examples, the breathing pattern detector 220 uses the values of the breathing phase time period(s) 218 to generate the breathing pattern metric(s) 222 (e.g., in substantially real-time as the breathing phase time period data is generated). For example, the breathing pattern detector 220 calculates the respiration frequency f_{R} for a breathing cycle based on the values of Tᵢ, Tᵢₚ, Tₑ, Tₑₚ. The breathing pattern detector 220 calculates the duration of each breathing phase relative to the total duration of the breathing cycle (e.g., Tᵢ*f_{R}). In the example of FIG. 5, the breathing phase time period(s) 218 and/or the breathing pattern metric(s) 222 represent breathing patterns in the nasal bridge vibration data 112.

The example breathing activity detector 224 applies one or more breathing pattern rule(s) 226 to associate the breathing pattern metric(s) 222 with the one or more activities (e.g., smelling, quiet breathing). For example, the breathing activity detector 224 analyzes the breathing pattern metric(s) 222 based on a rule indicating that during smelling, the inspiration pause and the expiration pause have decreased duration relative to quiet breathing.

In other examples, the breathing phase time period(s) 218 are analyzed using the machine learning-based classifier 230 that automatically associates the breathing phase time period(s) 218 with breathing activities based on previous training and machine learning algorithms. In some examples, the trainer 232 trains the classifier 230 to recognize the breathing activities based on the breathing phase(s) 212, the breathing phase time period(s) 218, the breathing pattern metric(s) 222, and/or the breathing activity classification(s) 228 generated for previously collected breathing data (e.g., nasal bridge vibration data 112).

In the example of FIG. 5, the breathing activity detector 224 or the classifier 230 generate the breathing activity classification(s) 228 that identify the breathing activities associated with the breathing patterns in the breathing data (e.g., as represented by the breathing phase time period(s) 218, the breathing pattern metric(s) 222) (block 512). The breathing activity classification(s) 228 indicate that the nasal bridge vibration data 112 collected at a given time is associated with the activities of quiet breathing, smelling, breathing during exercise, etc.

In examples where the breathing activity classification(s) 228 are generated by the machine learning-based classifier 230, the post-processing engine 234 verifies the classification(s) based on verification rule(s) 236 (block 514). The post-processing engine 234 detect and corrects error(s) in the classification(s) 228 based on the rule(s), such as different classifications assigned by the classifier 230 to adjacent breathing cycles. In such examples, the post-processing engine 234 determines if the classifier should be trained (i.e., re-trained) if the error(s) exceed a threshold (block 516). If the classifier 230 is to be trained, the trainer 232 trains the classifier 230 based on, for example, the breathing phase(s) 212, the breathing pattern metric(s) 222, etc.

The alert generator 238 of the breathing pattern analyzer 122 activates one or more output devices (e.g., the wearable device 102, the wearable or non-wearable user device 118) to generate alert(s) 240 based on the breathing activity classification(s) 228 (block 518). The alert(s) 240 can include visual, audio, and/or tactical alerts that provide recommendations with respect to the user's breathing, breathing data monitoring, etc. The communicator 242 can transmit instructions for the output device(s) to generate the alert(s) 240 for presentation to the user and/or a third party.

The example breathing pattern analyzer 122 continues to analyze the breathing data 112 with respect to breathing phase(s) 212, breathing phase time period(s) 218, breathing pattern metric(s) 222, etc. (block 520). If there is no further breathing data to be analyzed, the instructions of FIG. 5 end (block 522).

FIG. 6 is a block diagram of an example processor platform 600 capable of executing the instructions of FIG. 5 to implement the example breathing pattern analyzer 122 of FIGS. 1 and/or 2. The processor platform 600 can be, for example, a server, a personal computer, a mobile device (e.g., a cell phone, a smart phone, a tablet such as an iPad™), a personal digital assistant (PDA), an Internet appliance, wearable device such as eyeglasses, or any other type of computing device.

The processor platform 600 of the illustrated example includes a processor 122. The processor 122 of the illustrated example is hardware. For example, the processor 122 can be implemented by one or more integrated circuits, logic circuits, microprocessors or controllers from any desired family or manufacturer. The hardware processor may be a semiconductor based (e.g., silicon based) device. In this example, the processor implements the breathing pattern analyzer and its components (e.g., the example A/D converter 202, the example filter 204, the example signal envelope calculator 206, the example breathing phase detector 210, phase timing calculator 216, the example breathing pattern detector 220, the example breathing activity detector 224, the example classifier 230, the example trainer 232, the example post-processing engine 234, the example alert generator 238, the example communicator 242). However, in some examples, one or more of the elements are implemented by a device other than the processor (e.g., a discrete A/D converter, a separate filter, etc.).

The processor 122 of the illustrated example includes a local memory 613 (e.g., a cache). The processor 122 of the illustrated example is in communication with a main memory including a volatile memory 614 and a non-volatile memory 616 via a bus 618. The volatile memory 614 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 616 may be implemented by flash memory and/or any other desired type of memory device. Access to the main memory 614, 616 is controlled by a memory controller. The database 200 of the breathing pattern analyzer may be implemented by the main memory 614, 616.

The processor platform 600 of the illustrated example also includes an interface circuit 620. The interface circuit 620 may be implemented by any type of interface standard, such as an Ethernet interface, a universal serial bus (USB), and/or a PCI express interface.

In the illustrated example, one or more input devices 622 are connected to the interface circuit 620. The input device(s) 622 permit(s) a user to enter data and/or commands into the processor 122. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 624 are also connected to the interface circuit 620 of the illustrated example. The output devices 624 can be implemented, for example, by display devices (e.g., a light emitting diode (LED), an organic light emitting diode (OLED), a liquid crystal display, a cathode ray tube display (CRT), a touchscreen, a tactile output device, a printer and/or speakers). The interface circuit 620 of the illustrated example, thus, typically includes a graphics driver card, a graphics driver chip and/or a graphics driver processor. Alerts of the alert generator 238 may be used to drive one or more of the output devices.

The interface circuit 620 of the illustrated example also includes a communication device such as a transmitter, a receiver, a transceiver, a modem and/or network interface card to facilitate exchange of data with external machines (e.g., computing devices of any kind) via a network 626 (e.g., an Ethernet connection, a digital subscriber line (DSL), a telephone line, coaxial cable, a cellular telephone system, etc.). Alerts of the alert generator may be transmitted to another device outside the processor platform via the interface circuit 620.

The processor platform 600 of the illustrated example also includes one or more mass storage devices 628 for storing software and/or data. Examples of such mass storage devices 628 include floppy disk drives, hard drive disks, compact disk drives, Blu-ray disk drives, RAID systems, and digital versatile disk (DVD) drives.

The coded instructions 632 of FIG. 5 may be stored in the mass storage device 628, in the volatile memory 614, in the non-volatile memory 616, and/or on a removable tangible computer readable storage medium such as a CD or DVD.

From the foregoing, it will be appreciated that methods, systems, and apparatus have been disclosed to identify breathing activities associated with different breathing patterns. Disclosed examples analyze breathing data. The breathing data may be nasal bridge vibration data collected from a user wearing a wearable device such as eyeglasses including piezoelectric sensors to detect nasal bridge vibrations. In some examples, the analysis is performed at the wearable device, at a user device such as a smartphone, and/or via a cloud-based device. Disclosed examples identify breathing phases based on features of the breathing data (e.g., the inspiration phase, the inspiration pause, the expiration phase, the expiration pause, etc.). Some disclosed examples analyze all four of the phases (inspiration, inspiration pause, expiration, expiration pause) and, thus, account for a full breathing cycle including pauses between inhaling and exhaling air.

Disclosed examples calculate durations of corresponding breathing phase(s) and use the breathing phase timing information to classify the breathing data as associated with one or more breathing activities such as smelling, quiet breathing, etc. Some disclosed examples generate breathing pattern metrics based on the breathing phase time periods and classify the breathing data based on rules associated with the metrics. Other disclosed examples use machine learning algorithms to identify the breathing activities based on the breathing phase time periods. Disclosed examples generate customized alerts based on the breathing activity classifications, such as alerts when the user's breathing pattern change, recommendations for the user to adjust his or her breathing based on the activity, etc.

The following is a non-exclusive list of examples disclosed herein. Other examples may be included above. In addition, any of the examples disclosed herein can be considered in whole or in part, and/or modified in other ways.

Example 1 includes a wearable device including a sensor positioned to generate vibration signal data from a nasal bridge of a user; a breathing phase detector to identify a first breathing phase and a second breathing phase based on the vibration signal data; a phase timing calculator to calculate a first time period for the first breathing phase and a second time period for the second breathing phase; a breathing pattern detector to generate a breathing pattern metric based on the first time period and the second time period; a breathing activity detector to identify a breathing activity associated with the vibration signal data based on the breathing pattern metric; and an alert generator to activate an output device to generate at least one of an audible, tactile, or visual alert based on at least one of the breathing activity and a change associated with the breathing activity.

Example 2 includes the wearable device as defined in example 1, wherein the breathing phase detector is to identify the first breathing phase based on an amplitude level of the vibration signal data relative to a breathing phase threshold.

Example 3 includes the wearable device as defined in example 2, wherein the first breathing phase is one of an inspiration phase, an inspiration pause, an expiration phase, or an expiration pause.

Example 4 includes the wearable device as defined in examples 1 or 2, wherein the breathing activity is one of smelling, quiet breathing, or breathing during exercise by the user.

Example 5 includes the wearable device as defined in examples 1 or 2, further including a signal envelope calculator to calculate a signal envelope for the vibration signal data. In example 5, the breathing phase detector is to identify the first breathing phase and the second breathing phase based on the signal envelope.

Example 6 includes the wearable device as defined in example 5, wherein the breathing phase detector is to identify the first breathing phase and the second breathing phase based on an amplitude change in the signal envelope.

Example 7 includes the wearable device as defined in example 1, wherein the first breathing phase is an inspiration phase and the second breathing phase is an expiration phase, and the breathing phase detector is to further identify an inspiration pause and an expiration pause based on the vibration signal data.

Example 8 includes wearable device as defined in example 1, wherein the alert includes an instruction for the user to adjust at least one of their breathing rate or their activity.

Example 9 includes wearable device as defined in examples 1, 2, or 7, further including a filter to filter the vibration signal data based on a frequency band corresponding to nasal breathing.

Example 10 includes the wearable device as defined in example 9, wherein the breathing phase detector is to detect a change in the amplitude in a portion of the vibration signal data and associate the portion with oral breathing.

Example 11 includes the wearable device as defined in examples 1, 2, or 7, further including a communicator to transmit instructions to the output device to generate the alert.

Example 12 includes the wearable device as defined in examples 1, 2, or 7, wherein the output device is a non-wearable user device.

Example 13 includes the wearable device as defined in example 1, wherein the first breathing phase and the second breathing phase are associated with a first breathing cycle and the breathing phase detector is to identify a third breathing phase and a fourth breathing phase for a second breathing cycle based on the vibration signal data.

Example 14 includes the wearable device as defined in example 13, wherein the breathing activity detector is to identify a first breathing activity associated with the first breathing cycle and a second breathing activity associated with the second breathing cycle.

Example 15 includes the wearable device as defined in example 14, wherein the breathing pattern metric is a first breathing pattern metric and the breathing pattern detector is to generate a second breathing pattern metric based on the third breathing phase and the fourth breathing phase. In example 16, the breathing activity detector is to detect a change between the first breathing pattern metric and the second breathing metric and identify the second breathing activity based on the second breathing pattern metric and the change.

Example 16 includes at least one non-transitory computer readable storage medium including instructions that, when executed, cause a machine to identify a first breathing phase and a second breathing phase based on vibration signal data generated via a sensor from a nasal bridge of a user; calculate a first time period for the first breathing phase and a second time period for the second breathing phase; generate a breathing pattern metric based on the first time period and the second time period; identify a breathing activity associated with the vibration signal data based on the breathing pattern metric; and activate an output device to generate at least one of an audible, tactile, or visual alert based on at least one of the breathing activity and a change associated with the breathing activity.

Example 17 includes the at least one non-transitory computer readable storage medium as defined in example 16, wherein the instructions further cause the machine to identify the first breathing phase based on an amplitude level of the vibration signal data relative to a breathing phase threshold.

Example 18 includes the at least one non-transitory computer readable storage medium as defined in examples 16 or 17, wherein the instructions further cause the machine to calculate a signal envelope for the vibration signal data and identify the first breathing phase and the second breathing phase based on the signal envelope.

Example 19 includes the at least one non-transitory computer readable storage medium as defined in example 16, wherein the first breathing phase is an inspiration phase and the second breathing phase is an expiration phase, and the instructions further cause the machine to identify an inspiration pause and an expiration pause based on the vibration signal data.

Example 20 includes the at least one non-transitory computer readable storage medium as defined in examples 16, 17, or 19, wherein the instructions further cause the machine to filter to filter the vibration signal data based on a frequency band corresponding to nasal breathing.

Example 21 includes the at least one non-transitory computer readable storage medium as defined in example 16, wherein the first breathing phase and the second breathing phase are associated with a first breathing cycle and the instruction further cause the machine to identify a third breathing phase and a fourth breathing phase for a second breathing cycle based on the vibration signal data.

Example 22 includes the at least one non-transitory computer readable storage medium as defined in example 21, wherein the instructions cause the machine to identify a first breathing activity associated with the first breathing cycle and a second breathing activity associated with the second breathing cycle.

Example 23 includes the at least one non-transitory computer readable storage medium as defined in example 22, wherein the breathing pattern metric is a first breathing pattern metric and wherein the instructions cause the machine to generate a second breathing pattern metric based on the third breathing phase and the fourth breathing phase; detect a change between the first breathing pattern metric and the second breathing metric; and identify the second breathing activity based on the second breathing pattern metric and the change.

Example 24 includes a method including identifying, by executing an instruction with a processor, a first breathing phase and a second breathing phase based on vibration signal data generated via a sensor from a nasal bridge of a user; calculating, by executing an instruction with the processor, a first time period for the first breathing phase and a second time period for the second breathing phase; generating, by executing an instruction with the processor, a breathing pattern metric based on the first time period and the second time period; identifying, by executing an instruction with the processor, a breathing activity associated with the vibration signal data based on the breathing pattern metric; and activating, by executing an instruction with the processor, an output device to generate at least one of an audible, tactile, or visual alert based on at least one of the breathing activity and a change associated with the breathing activity.

Example 25 includes the method as define in example 24, wherein the first breathing phase is one of an inspiration phase, an inspiration pause, an expiration phase, or an expiration pause.

Example 26 includes method as defined in example 24, further including calculating a signal envelope for the vibration signal data and identifying the first breathing phase and the second breathing phase based on the signal envelope.

Example 27 includes an apparatus including a breathing phase detector to identify a first breathing phase and a second breathing phase based on first vibration signal data collected from a nasal bridge of a user; a breathing pattern detector to generate a breathing pattern metric based on the first breathing phase and the second breathing phase; a classifier to assign a breathing activity classification to second vibration signal data collected from the user or a nasal bridge of another user; and a trainer to train the classifier to assign the breathing activity classification to the second vibration signal data without the breathing pattern detector generating the breathing pattern metric for the second vibration data.

Example 28 includes the apparatus as defined in example 27, wherein the trainer is to train the classifier based on a signal envelope profile for the first vibration signal data.

Example 29 includes the apparatus as defined in example 27, wherein the trainer is to train the classifier based on one or more of (1) the first breathing phase for the first vibration signal data, (2) the second breathing phase for the first vibration signal data, (3) the breathing pattern metric for the first vibration signal data, (4) one or more breathing phase time periods for the first vibration signal data, and (5) one or more breathing activity classifications for the first vibration signal data.

Example 30 includes the apparatus as defined in example 27, further including a post-processing engine to verify the breathing activity classification.

Example 31 includes the apparatus as defined in example 30, wherein the post-processing engine is to determine if the trainer is to be re-trained based on the verification.

Example 32 includes the apparatus as defined in example 30, wherein if the post-processing engine detects an error in the breathing activity classification, the post-processing engine is to correct the error.

Example 33 includes an apparatus including means for identifying a breathing cycle in vibration data obtained from a nasal bridge of a user, the breathing cycle including a first breathing phase, a second breathing phase, a third breathing phase, and a fourth breathing phase; means for determining respective durations of each of the first breathing phase, the second breathing phase, the third breathing phase, and the fourth breathing phase; means for identifying a breathing activity associated with the breathing cycle based on the respective durations; and means for generating an alert based on the identification of the breathing activity.

Example 34 includes the apparatus of example 33, further including means for transmitting the alert to an output device.

Example 35 includes the apparatus of example 33, further including means for training the means for identifying the breathing activity to automatically identify the breathing activity.

Example 36 includes the apparatus of example 33, wherein the means for training includes a trainer to train the means for identifying the breathing activity based on a machine-learning algorithm.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A wearable device comprising:
a sensor positioned to generate vibration signal data from a nasal bridge of a user;
a breathing phase detector to identify a first breathing phase and a second breathing phase based on the vibration signal data;
a phase timing calculator to calculate a first time period for the first breathing phase and a second time period for the second breathing phase;
a breathing pattern detector to generate a breathing pattern metric based on the first time period and the second time period;
a breathing activity detector to identify a breathing activity associated with the vibration signal data based on the breathing pattern metric; and
an alert generator to activate an output device to generate at least one of an audible, tactile, or visual alert based on at least one of the breathing activity and a change associated with the breathing activity.

2. The wearable device as defined in claim 1, wherein the breathing phase detector is to identify the first breathing phase based on an amplitude level of the vibration signal data relative to a breathing phase threshold.

3. The wearable device as defined in claim 2, wherein the first breathing phase is one of an inspiration phase, an inspiration pause, an expiration phase, or an expiration pause.

4. The wearable device as defined in claim 1, wherein the breathing activity is one of smelling, quiet breathing, or breathing during exercise by the user.

5. The wearable device as defined in claim 1, further including a signal envelope calculator to calculate a signal envelope for the vibration signal data, the breathing phase detector to identify the first breathing phase and the second breathing phase based on the signal envelope.

6. The wearable device as defined in claim 5, wherein the breathing phase detector is to identify the first breathing phase and the second breathing phase based on an amplitude change in the signal envelope.

7. The wearable device as defined in claim 1, wherein the first breathing phase is an inspiration phase and the second breathing phase is an expiration phase, and the breathing phase detector is to further identify an inspiration pause and an expiration pause based on the vibration signal data.

8. The wearable device as defined in claim 1, wherein the alert includes an instruction for the user to adjust at least one of their breathing rate or their activity.

9. The wearable device as defined in claim 1, further including a filter to filter the vibration signal data based on a frequency band corresponding to nasal breathing.

10. At least one non-transitory computer readable storage medium comprising instructions that, when executed, cause a machine to:
identify a first breathing phase and a second breathing phase based on vibration signal data generated via a sensor from a nasal bridge of a user;
calculate a first time period for the first breathing phase and a second time period for the second breathing phase;
generate a breathing pattern metric based on the first time period and the second time period;
identify a breathing activity associated with the vibration signal data based on the breathing pattern metric; and
activate an output device to generate at least one of an audible, tactile, or visual alert based on at least one of the breathing activity and a change associated with the breathing activity.

11. The at least one non-transitory computer readable storage medium as defined in claim 10, wherein the instructions further cause the machine to identify the first breathing phase based on an amplitude level of the vibration signal data relative to a breathing phase threshold.

12. The at least one non-transitory computer readable storage medium as defined in claim 10, wherein the instructions further cause the machine to:
calculate a signal envelope for the vibration signal data; and
identify the first breathing phase and the second breathing phase based on the signal envelope.

13. The at least one non-transitory computer readable storage medium as defined in claim 10, wherein the first breathing phase is an inspiration phase and the second breathing phase is an expiration phase, and the instructions further cause the machine to identify an inspiration pause and an expiration pause based on the vibration signal data.

14. The at least one non-transitory computer readable storage medium as defined in claim 10, wherein the instructions further cause the machine to filter to filter the vibration signal data based on a frequency band corresponding to nasal breathing.

15. The at least one non-transitory computer readable storage medium as defined in claim 10, wherein the first breathing phase and the second breathing phase are associated with a first breathing cycle and the instruction further cause the machine to identify a third breathing phase and a fourth breathing phase for a second breathing cycle based on the vibration signal data.

16. The at least one non-transitory computer readable storage medium as defined in claim 15, wherein the instructions cause the machine to identify a first breathing activity associated with the first breathing cycle and a second breathing activity associated with the second breathing cycle.

17. The at least one non-transitory computer readable storage medium as defined in claim 16, wherein the breathing pattern metric is a first breathing pattern metric and wherein the instructions cause the machine to:
generate a second breathing pattern metric based on the third breathing phase and the fourth breathing phase;
detect a change between the first breathing pattern metric and the second breathing metric; and
identify the second breathing activity based on the second breathing pattern metric and the change.

18. A method comprising:
identifying, by executing an instruction with a processor, a first breathing phase and a second breathing phase based on vibration signal data generated via a sensor from a nasal bridge of a user;
calculating, by executing an instruction with the processor, a first time period for the first breathing phase and a second time period for the second breathing phase;
generating, by executing an instruction with the processor, a breathing pattern metric based on the first time period and the second time period;
identifying, by executing an instruction with the processor, a breathing activity associated with the vibration signal data based on the breathing pattern metric; and
activating, by executing an instruction with the processor, an output device to generate at least one of an audible, tactile, or visual alert based on at least one of the breathing activity and a change associated with the breathing activity.

19. The method as define in claim 18, wherein the first breathing phase is one of an inspiration phase, an inspiration pause, an expiration phase, or an expiration pause.

20. The method as defined in claim 18, further including:
calculating a signal envelope for the vibration signal data; and
identifying the first breathing phase and the second breathing phase based on the signal envelope.
